(19) United States
(12) Patent Application Publication
(10) Pub. No.: US 2011/0183369 A1
(43) Pub. Date: Jul. 28, 2011

(54) PLANT INFESTING SYSTEMS AND METHODS

(75) Inventors: Peter L. Clark, St. Peters, MO (US); Claudia Gianni, Wildwood, MO (US); Joel Tindle, Steward, IL (US)

(73) Assignee: MONSANTO TECHNOLOGY LLC, St. Louis, MO (US)

(21) Appl. No.: 13/014,068

(22) Filed: Jan. 26, 2011

Related U.S. Application Data
(60) Provisional application No. 61/299,120, filed on Jan. 28, 2010.

(51) Int. Cl. C12Q 1/02; C12M 1/00

(52) U.S. Cl. 435/29; 435/283.1

(57) ABSTRACT

The present disclosure provides systems and methods for infesting the roots of a plant with larval insects. In various embodiments, an exemplary method includes injecting an egg solution into a root zone of the plant, wherein the root zone is disposed within a planting media of the plant.

### CROSS-REFERENCE TO RELATED APPLICATIONS

This application claims the benefit of U.S. Provisional Application No. 61/299,120, filed on Jan. 28, 2010, the disclosure of which is incorporated herein by reference in its entirety

### FIELD

The present teachings relate to systems and methods for screening plants for pest tolerance.

### BACKGROUND

The statements in this section merely provide background information related to the present disclosure and may not constitute prior art.

In plant breeding and selection processes, genotypic and/or phenotypic data can be gathered from insect infested plants to determine whether particular plants, e.g., corn plants, are resistant or susceptible to one or more particular insects, e.g., western corn rootworm. Additionally, such genotypic and/or phenotypic data can be gathered from infested plants to screen and select plants that possess a particular genetic trait that are resistant to particular insects. For example, by infesting corn plants with western corn rootworm, the efficacy of rootworm resistant genetic traits of various insecticidal hybrid and/or inbred corn plants can be tested.

Known methods and systems for infesting plants are typically unable to produce a consistent level of infestation and, therefore, yield inconsistent and/or inaccurate data. For example, one known system and method utilizes a vehicle to pull a blade through the ground along a row of plants to create a furrow adjacent the row of plants. A solution comprising insect eggs is then deposited into the furrow. Subsequently, the eggs hatch and the larvae attempt to migrate from the furrow location to the root zone of the plants in the row, thereby infesting the plants. However, the mortality rate of the larvae as they migrate through the ground can cause the occurrence and/or levels of plant infestation to be inconsistent such that the resulting data can be inconsistent and/or inaccurate. Additionally, occasionally the furrow can run too close to the plants and/or too deep such that the blade cuts or damages the roots of the plants, thereby resulting in inconsistent and/or inaccurate data.

### SUMMARY

The present disclosure provides systems and methods for infesting the roots of a plant with larval insects.

In various embodiments, an exemplary method includes injecting an egg solution into a root zone of the plant, wherein the root zone is disposed within a planting media of the plant.

In various other embodiments, an exemplary method includes injecting an egg solution into a root zone of the plant, wherein the root zone is disposed within a planting media of the plant. Moreover, in such embodiments, the egg solution includes a plurality of insect eggs suspended in a solution, wherein the egg solution has a first portion of eggs that have been incubated for a first period of time and a second portion of eggs that have been incubated for a second period of time.

Still yet in various other embodiments, an exemplary method includes depositing a quantity of an egg solution in an egg solution supply container of an automated egg solution injection system. The egg solution includes a plurality of insect eggs suspended in a solution, wherein a first portion of eggs have been incubated for a first period of time and a second portion of eggs that have been incubated for a second period of time. The method additionally includes creating at least one pilot hole in a planting media from which the plant is growing such that the at least one pilot hole extends into a root zone of the plant, wherein the root zone is disposed within the planting media. Furthermore, the method includes placing a tip of an injection gun dispensing wand of the automated egg solution injection system in close proximity to a base of the at least one pilot hole at a surface of the planting media. The injection gun and dispensing wand are fluidly connected to the egg solution supply container. The method still further includes dispensing a quantity of the egg solution from the tip of the dispensing wand into the at least one pilot hole at a regulated pressure using a pressure regulator of the automated egg solution injection system such that the egg solution is injected into the at least one pilot hole and at least a substantial portion of the injected egg solution is deposited in the root zone.

In various other embodiments, an exemplary plant root infestation system is provided that includes a hole punch device structured and operable to create at least one pilot hole in a planting media from which the plant is growing such that the at least one pilot hole extends into a root zone of the plant. The system additionally includes an automated egg solution injection system structured and operable to inject an egg solution into the at least one pilot hole such that at least a substantial portion of the egg solution is deposited into the root zone of the plant. The egg solution includes a plurality of insect eggs suspended in a solution, wherein the eggs comprise a first portion that have been incubated for a first period of time and a second portion that have been incubated for a second period of time.

In various implementations, the automated egg solution injection system comprises at least one egg solution supply container structured to retain a quantity of the egg solution and a propellant container structured to retain a quantity of pressurized gas. The automated egg solution injection system additionally includes a pressure regulator coupled to the propellant container and fluidly connected to the at least one egg solution supply container via a gas conduit. The pressure regulator is structured and operable to release the pressurized gas from the propellant container at a regulated pressure, where after the released gas is directed into the at least one egg solution supply container via the gas conduit. The automated egg solution injection system further includes an injection gun fluidly connected to the at least one egg solution supply container via a solution hose. The injection gun is structured and operable to receive the egg solution forced from the at least one egg solution supply container at the regulated pressure by the gas directed from the propellant container into the at least one egg solution supply container at the regulated pressure, and controllably dispense the egg solution at the regulated pressure through a nozzle of the injection gun upon activation of a trigger mechanism of the injection gun.

Still further, the automated egg solution injection system includes a dosage regulator coupled to the injection gun nozzle. The dosage regulator is structured and operable to receive the egg solution from the injection gun nozzle and dispense a metered amount of the egg solution through an outlet of the dosage regulator. The egg solution injection system still yet further includes a dispensing wand coupled to the dosage regulator outlet. The dispensing wand is structured to convey the egg solution from the dosage regulator outlet to a tip of the dispensing wand. Subsequently, when the tip of the dispensing wand is place in closed proximity to a base of the hole at the surface of the planting media, the metered amount of egg solution is dispensed from the wand tip at the regulated pressure such that egg solution is injected into the at least one pilot hole and deposited in the root zone.

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is an isometric view of a plant root infestation system, in accordance with various embodiments of the present disclosure.

FIG. 2 is a schematic view of a plant having a root zone that can be infested with larval insects utilizing the plant root infestation system shown in FIG. 1, in accordance with various embodiments of the present disclosure.

FIG. 3 is an isometric view of a propellant container and a pressure regulator of the plant root infestation system shown in FIG. 1, in accordance with various embodiments of the present disclosure.

FIG. 4 is an isometric view of an injection gun and dosage regulator of the plant root infestation system shown in FIG. 1, in accordance with various embodiments of the present disclosure.

FIG. 5 is a flow chart illustrating the operation of the plant root infestation system shown in FIG. 1 to infest the roots of a plant with larval insects, in accordance with various embodiments of the present disclosure.

FIG. 6 is an isometric view of a portion of a hole punch device of the plant root infestation system shown in FIG. 1, in accordance with various embodiments of the present disclosure.

FIG. 7 is a schematic view of a plant having a root zone that can be infested with larval insects utilizing the plant root infestation system shown in FIG. 1, in accordance with various embodiments of the present disclosure.

FIG. 8 is a graphic illustration of test infestation results of corn plants utilizing the plant root infestation system shown in FIG. 1, in accordance with various embodiments of the present disclosure.

FIG. 9 is a graphic illustration of a comparison of infestation results of corn plants between known infestation means and the plant root infestation system shown in FIG. 1, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

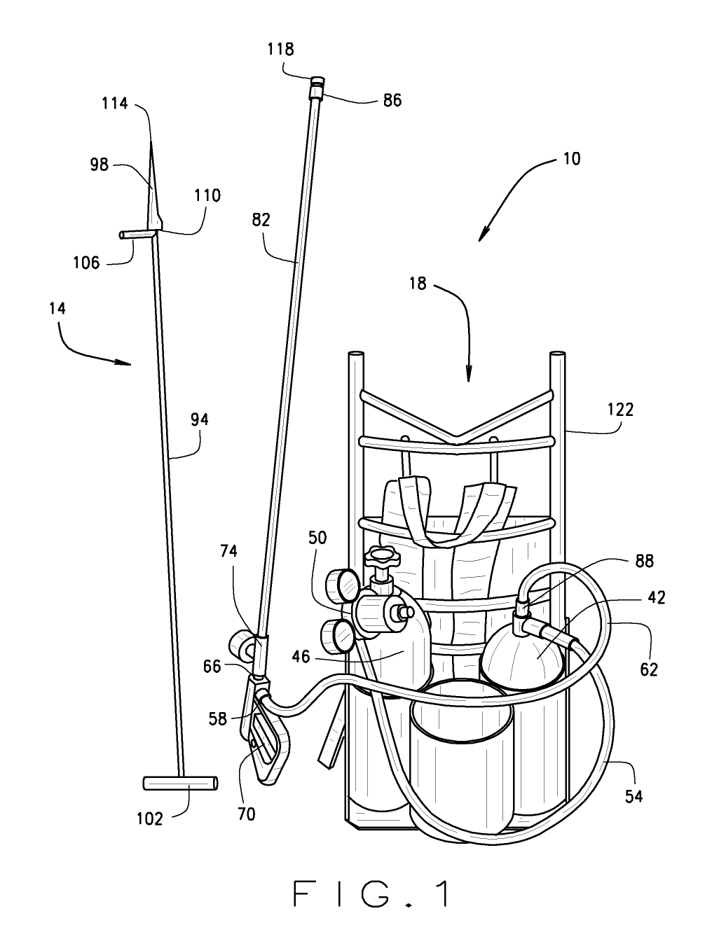

Referring to FIG. 1, in various embodiments, the present disclosure provides a plant root infestation system 10 that is structured and operable to infest the roots of a plant with larval insects. More particularly, the plant root infestation system 10, as disclosed herein, can be utilized to infest the roots of a plurality of plants at a high throughput rate with a substantially high rate of infestation and a substantially consistent level of infestation for all the plants. For example, the plant root infestation system 10 can be employed to infest the roots of an entire test plot of corn plants with western corn rootworm eggs at a high throughput rate with a substantially high rate of infestation and a substantially consistent level of infestation for all the corn plants.

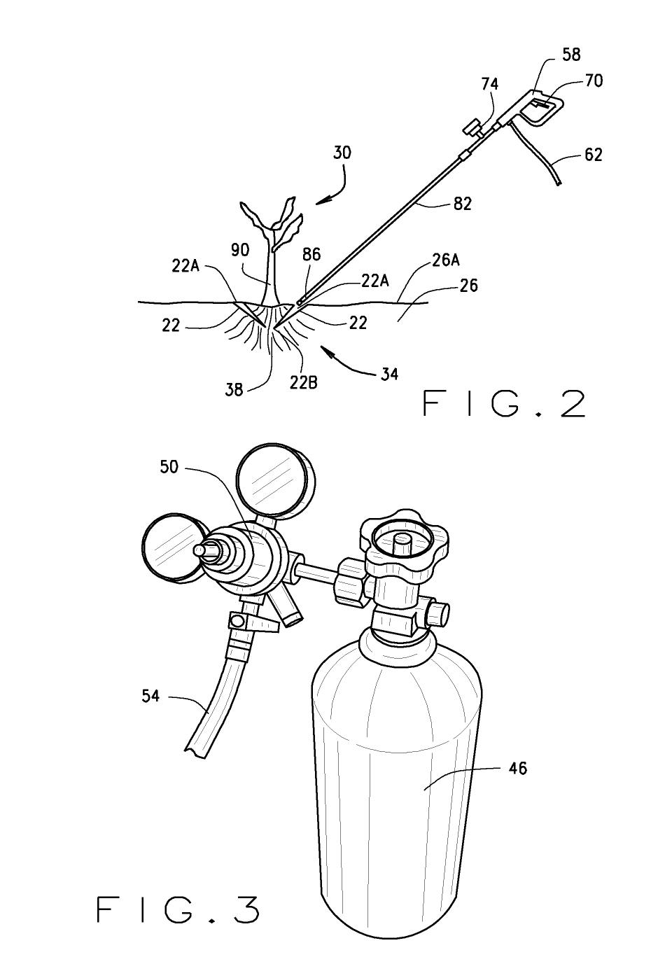

Referring now to FIGS. 1 and 2, in various embodiments, the plant root infestation system 10 includes a hole punch device 14 and an automated egg solution injection system 18. The hole punch device 14 is structured and operable to create at least one pilot hole 22 in a planting media 26, e.g. soil, from which a plant 30 is growing such that the pilot hole(s) 22 extend(s) into a root zone 34 of the plant 30, wherein the root zone 34 includes roots 38 of the plant 30 that are disposed, i.e., growing, within the planting media 26. The automated egg solution injection system 18 is structured and operable to inject an egg solution into the pilot hole(s) 22 such that at least a substantial portion of the egg solution is deposited into the root zone 34 of the plant 30. The egg solution comprises a plurality of insect eggs, e.g., western corn rootworm eggs, disposed in, or mixed with, an egg carrying or suspension solution or media, e.g., a viscous or agar solution or media. As described above, the plant root infestation system 10 can be utilized to infest the roots 38 of a plurality of plants 30. Hence, although only a single plant 30 is illustrated in the various figures, it should be understood that the plant root infestation system 10 can be utilized to infest the roots 38 of a plurality of plants 30. And, more particularly, the plant root infestation system 10 can be utilized to infest the roots 38 of a plurality of plants 30 at a high throughput rate with a substantially high rate of infestation and a substantially consistent level of infestation for all the plants.

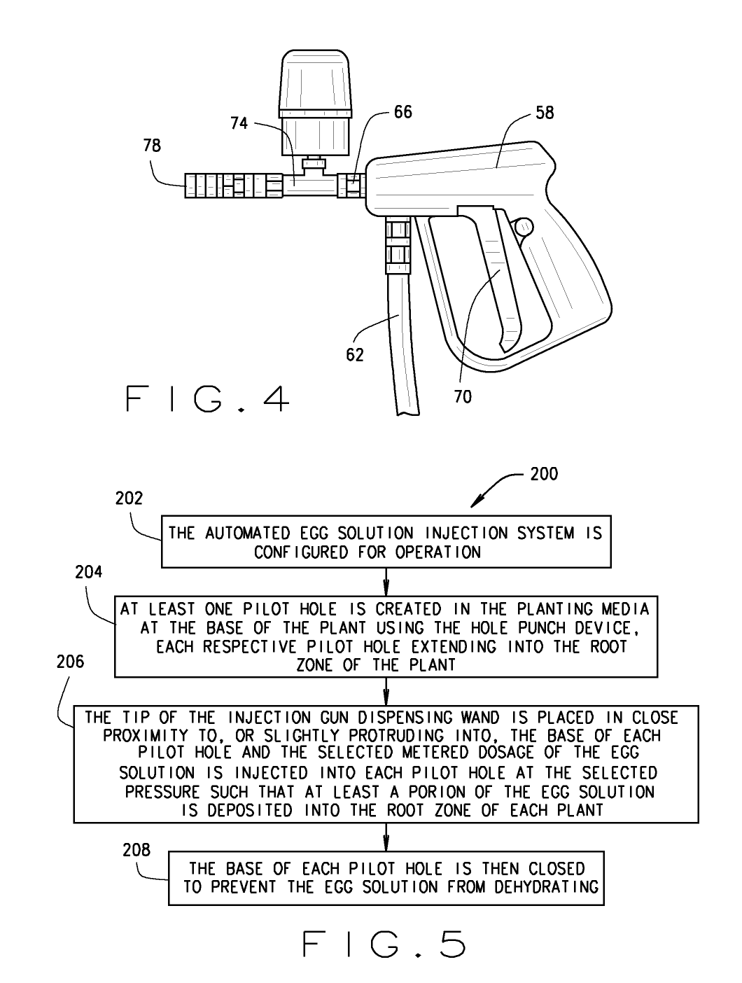

Referring additionally to FIGS. 3 and 4, in various embodiments, the automated egg solution injection system 18 includes at least one egg solution supply container 42 structured to retain a quantity, e.g., 3 liters or more, of the egg solution, and at least one propellant container 46 structured to retain a quantity of pressurized gas propellant, e.g., pressurized CO₂. The automated egg solution injection system 18 can additionally include a pressure regulator 50 coupled to the propellant container(s) 46 and fluidly connected to the egg solution supply container(s) 42 via a gas conduit 54, e.g., flexible pressure tubing or other pressure rated tubing or piping. The pressure regulator 50 is structured and operable to control the release of pressurized gas from the propellant container(s) 46 such that the pressurized gas is released at a regulated pressure. More particularly, the gas is released from propellant container(s) 46 at a selectable regulated pressure, e.g., 15-30 psi, and directed into the egg solution supply container(s) 42 via the gas conduit 54.

In various embodiments, the automated egg solution injection system 18 additionally includes an injection gun 58 that is fluidly connected to the egg solution supply container(s) 42 via a solution hose 62, e.g., flexible hosing or tubing. The injection gun 58 is structured and operable to receive, via the solution hose 62, the egg solution forced from the egg solution supply container(s) 42 at the selected regulated pressure by the gas directed from the propellant container(s) 46 into the egg solution supply container(s) 42. Moreover, the injection gun 58 is structured and operable to controllably dispense the received egg solution at the selected regulated pressure through a nozzle 66 of the injection gun 58 upon activation, e.g., depression, of a trigger mechanism 70 of the injection gun 58. In various embodiments, the automated egg solution injection system 18 includes a dosage regulator 74 coupled to the injection gun nozzle 66. The dosage regulator 74 is structured and operable to receive the egg solution from the injection gun nozzle 66 and dispense a selectable metered amount, e.g., 5 ml, of the egg solution through an outlet 78 of the dosage regulator 74.

In various embodiments, the automated egg solution injection system 18 further includes a dispensing wand 82 coupled to the dosage regulator outlet 78. The dispensing wand 82 is structured to convey, or carry, the egg solution from the dosage regulator outlet 78 to a tip 86 of the dispensing wand 82, whereby the selected metered amount of egg solution is dispensed from the wand tip 86 at the selected regulated pressure. More specifically, prior to activation of the injection gun trigger mechanism 70, the wand tip 86 can be placed in close proximity to, or slightly protruding into, a base 22A of a pilot hole 22 at a surface 26A of the planting media 26 such that upon activation of the injection gun trigger mechanism 70, the metered dosage of egg solution is injected into the respective pilot hole 22 at the regulated pressure and deposited in the root zone 34.

FIG. 5 provides a flow chart 200 illustrating the operation of the plant root infestation system 10, as described herein, to infest the roots 38 of one or more plants 30, e.g., one or more stage V2 or V3 corn plants, with larval insects, e.g., western corn rootworm. In various embodiments, initially the automated egg solution injection system 18 is configured for operation, as indicated at 202. To configure the automated egg solution injection system 18 for operation, a quantity, e.g., 3 liters, of the egg solution is deposited into the egg solution container 42, via an inlet of the egg solution container 42, and the gas conduit 54 and solution hose 62 are connected to the egg solution container 42, via a coupling cap 88. The coupling cap 88 is structured and operable to provide a substantially air-tight seal to egg solution container inlet. The coupling cap 88 is further structured and operable to connect the gas conduit 54 to the egg solution container 42 such that gas propellant from the propellant container 46 is allowed to flow into the egg solution container 42 to pressurize the egg solution container 42, and to connect the solution hose 62 to the egg solution container 42 such that the egg solution is allowed to flow out of the egg solution container to the injection gun 58, via the solution hose 62, as a result of the provided gas pressure.

After the egg solution is deposited into the egg solution container 42 and the coupling cap 88 is connected, configuration of the automated egg solution injection system 18 further includes releasing the gas propellant from the propellant container 46 such that the gas propellant is allowed to flow though the gas conduit 54, as controlled and regulated by pressure regulator 50, into the egg solution container 42 at a selected pressure, or flow rate, e.g., 15-30 psi, whereby the gas propellant pressurizes the egg solution container 42 at the selected pressure.

Next, at least one pilot hole 22 is created in the planting media 26 at the base of each plant 30 a certain distance, e.g., 1 inch, from a stalk 90 of the respective plant 30 using the hole punch device 14. Importantly, each pilot hole 22 is created such that each respective pilot hole 22 extends into the root zone 34, i.e., into the roots 38, of the respective plant 30, as indicated at 204. It should be understood that the order of configuring the egg solution injection system 18 and then creating the pilot hole(s) 22 is merely exemplary and that the order could be reversed and remain within the scope of disclosure. That is, in various embodiments, the pilot hole(s) 22 can be created first and then the egg solution injection system 18 is configured. It should also be understood that it is envisioned that one, two or more pilot holes 22 can be created and remain within the scope of the present invention. Creating a plurality of pilot holes 22 around the stalk 90 of each plant 30 will increase the infestation rate for the respective plants 30. For example, in various embodiments, a pair of opposing pilot holes 22 are created, e.g., a first pilot hole 22 and a second pilot hole 22 on the opposing side of each plant stalk 90, wherein each pilot hole extends into the root zone 34 of the respective plant 30.

Once the egg solution injection system 18 has been configured and the pilot hole(s) 22 created, the tip 86 of the injection gun dispensing wand 82 is sequentially placed in close proximity to, or slightly protruding into, the base 22A of each pilot hole 22. The injection gun trigger mechanism 70 is then activated to dispense the selected metered dosage of the egg solution at the selected pressure into the respective pilot hole 22. More specifically, as a result of the selected pressure, the dispensed selected metered dosage is injected into the respective pilot hole 22 such that at least a portion, e.g., a substantial portion, of the egg solution is deposited into the root zone 34 of the respective plant 30, as indicated at 206. The base 22A of each pilot hole 22 is then closed to prevent the egg solution from dehydrating, e.g., by pushing some of the surrounding planting media 26 over the pilot hole base 22A, as indicated at 208.

Referring again to FIGS. 1 and 2, in various embodiments, the hole punch device 14 comprises a shaft 94 having a head 98 disposed at an end of the shaft 94 that is structured to create each pilot hole 22 by pushing the head 98 into the planting media 26 adjacent the plant stalk 90 such that the pilot hole 22 extends into the root zone 34 of the respective plant 30. In various embodiments, the hole punch device 14 can additionally include a handle 102 disposed at an end of the shaft 94 opposite the head 98 that is utilized to aid in pushing and guiding the head 98 into the planting media 26 to create each pilot hole 22. Furthermore, in various embodiments, the hole punch device 14 can include a depth gauge 106 extending substantially orthogonally from the shaft 94 at a top 110 of the head 98. The depth gauge 106 is structured to control the depth of each pilot hole 22. Particularly, the depth gauge extends from the shaft such that once the entire length of the head 98 is pushed into the planting media 26 such that the top 110 of the head 98 is substantially at the planting media surface 26A, the depth gauge 106 will contact the planting media surface 26A and prevent the head 98 from being pushed deeper into the planting media 26. Hence, the depth gauge 106 controls the depths of each pilot hole 22 such that each pilot hole 22 extends into the respective root zone 34, but does not extend through and beyond the respective root zone 34.

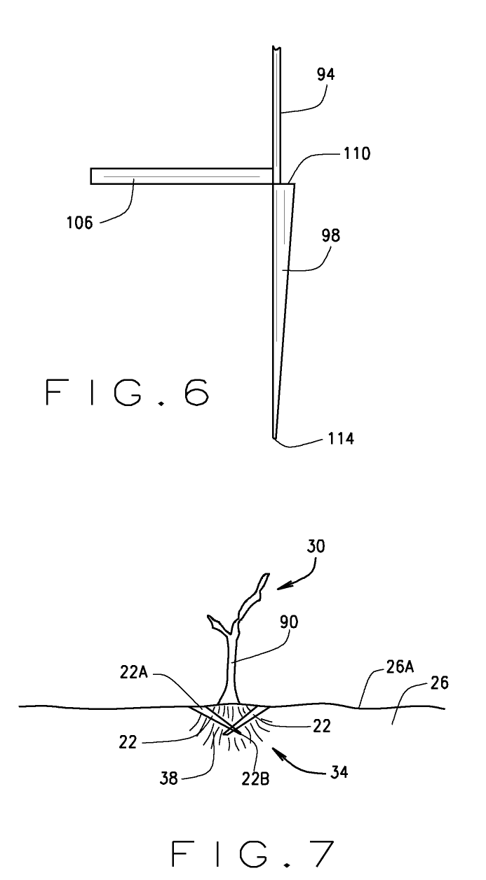

Referring now to FIG. 6, in various embodiments, the hole punch device head 98 is structure to have a conical shape such that each pilot hole 22 is created to have a corresponding conical shape. Therefore, the conical head 98 creates each pilot hole 22 such that each pilot hole base 22A will have a diameter that is larger than a diameter of a distal end 22B of the each respective pilot hole 22. Moreover, the conical shape of the hole punch device head 98 provides a substantially pointed tip 114 that allows the head 98 to be easily pushed into the planting media 26, and more particularly, allows the head 98 to pierce through the root zone 34 without damaging the roots 38 of the respective plant 30.

Referring now to FIGS. 2 and 7, as described above, each pilot hole 22 is created such that the respective pilot hole 22 extends into the root zone 34 of the plant 30 such that at least a portion of the injected egg solution will be deposited into the root zone 34. In various implementations, each pilot hole 22 can be create by inserting, or pushing, the hole punch device head 98 in to the planting media 26 at an angle. More specifically, the tip 114 of the hole punch device head 98 can be placed onto the planting media 26 a certain distance away from the stalk 90 of the respective plant 30, e.g., 2 inches. The head 98 can then be pushed downward and radially inward into the planting media 26 such that at least a distal portion of the resulting pilot hole 22, i.e., a portion of the pilot hole 22 away from the base 22A, is disposed within the root zone 34.

As also described above, in various implementations, a plurality of pilot holes 22 can be created around the plant stalk 90 to increase the infestation rate of the respective plant 30. For example, in various embodiments, a pair of the angled pilot holes 22 can be created on opposing sides of the respective plant stalk 90, whereby each angled pilot hole 22 extends into the respective root zone 34 such that the pair of pilot holes 22 substantially forms a V pattern, or shape, within the root zone 34, as illustrated in FIG. 2. Or, in various other embodiments, a pair of the angled pilot holes 22 can be created on opposing sides of the respective plant stalk 90, whereby each angled pilot hole 22 extends into the respective root zone 34 such that the pair of pilot holes 22 substantially forms a X pattern, or shape, within the root zone 34, as illustrated in FIG. 7.

Referring now to FIG. 1, in various embodiments, the dispensing wand 82 of the automated egg solution injection system 18 can include a check valve 118 coupled to the dispensing wand tip 86. The check valve 118 can be any uni-direction flow control device structured and operable to allow the selected metered dosage of egg solution to be dispensed from the dispensing wand 82, as described above, and to close, or seal, the dispensing wand tip 86 once the selected metered dosage has been dispensed. Particularly, the check valve 118 is structured and operable to close, or seal, the dispensing wand tip 86 such that the egg solution is prevented from leaking from of the dispensing wand tip 86 and loose planting media 26 is prevented from clogging the dispensing wand tip 86. For example, in various embodiments, the check valve 118 can comprise a spring biased ball check valve.

As described above, the egg solution comprises a plurality of insect eggs, e.g., western corn rootworm eggs, disposed in, or mixed with, an egg carrying, or suspension, solution or media, e.g., a viscous or agar solution or media. In various embodiments, the egg solution can be prepared to include a plurality of insect eggs, wherein a first portion of eggs have been incubated for a first period of time and a second portion of eggs that have been incubated for a second period of time. Accordingly, after the egg solution is injected into the pilot hole(s) 22, the first and second portions of eggs will hatch at different times, i.e., after differing time periods have elapsed. Having eggs of differing incubation periods and, hence, differing hatching times, increases the rate of infestation of each respective plant 30 and also provides a substantially consistent level of infestation for all plants 30 that have been infested via the plant root infestation system 10, as described herein.

For example, in various implementations, the egg solution can be prepared to include a first portion of eggs that have not been incubated and a second portion of eggs that have been incubated for five to six days. Thereafter, an entire test plot of plants 30 have the respective egg solution injected into the respective root zones 34, via the plant root infestation system 10, as described above. Subsequently, the first portion of eggs will hatch and the resulting larval insects will begin to attack the roots 38 of each plant 30 after a first period time. Then, after a second period, e.g., approximately five to six days after the first portion of eggs have hatched, the second portion of eggs will hatch and the resulting larval insects will begin to attack the roots 38 of each plant 30. The multi-tiered hatching, e.g., two-tiered hatching, as described above, will provide a longer period of larval insect activity in each respective root zone 34, and therefore, provide a high rate of infestation and also a substantially consistent level of infestation for all plants 30 in the test plot.

Although only a two-tiered hatching egg solution has been described, it should be understood that any multi-tiered hatching solution is envisioned and within the scope of the present disclosure. For example, the egg solution can be prepared to include a first, second and third, or more portion of eggs, i.e., a three-tier or more, wherein each respective portion of eggs has been incubated for a time period different than all of the other portions of eggs. Accordingly, the duration of time for which the roots 38 of each respective plant 30 is subjected to freshly hatched larval insects is increased, which will result in an increased rate and level of infestation for all plants 30 in the test plot. Moreover, the rate and level of infestation can be substantially controlled based on the number of portions of eggs, the concentration of eggs in the solution, e.g., 250 eggs/ml, and the respective incubation periods for each portion of eggs that are included in the egg solution.

Hence, in various embodiments, the operation of the plant root infestation system 10 can include utilizing an egg solution that includes a multi-tiered, i.e., a two-tier, three-tier or more, hatching solution. Referring again to FIG. 5, in such embodiments, operation of the plant root infestation system 10, to infest the roots 38 of one or more plants 30, e.g., one or more stage V2 or V3 corn plants, with larval insects, e.g., western corn rootworm, comprises configuring the automated egg solution injection system 18 for operation, as illustrated at 202, such that a multi-tiered hatching solution will be injected into the respective pilot hole(s) 22. More specifically, in such embodiments, configuring the automated egg solution injection system 18 comprises depositing a quantity, e.g., 3 liters, of a multi-tiered hatching egg solution into the egg solution supply container 42, wherein the multi-tiered hatching solution has been prepared to include a first portion of eggs that have been incubated for a first period of time and a second portion of eggs that have been incubated for a second period of time. In various implementations, either the first or the second periods of time can be substantially equal to zero seconds such that the respective portion of eggs are relatively non-incubated. As described above, the egg solution, i.e., the multi-tiered hatching egg solution, is deposited into the egg solution container 42, via the inlet of the egg solution container 42, and the gas conduit 54 and solution hose 62 are connected to the egg solution container 42, via the coupling cap 88.

After the multi-tiered hatching egg solution is deposited into the egg solution container 42 and the coupling cap 88 is connected, the gas propellant is released from the propellant container 46 such that the gas propellant is allowed to flow though the gas conduit 54, as controlled and regulated by the pressure regulator 50, into the egg solution container 42 at a selected pressure, or flow rate, e.g., 15-30 psi, whereby the gas propellant pressurizes the egg solution container 42 at the selected pressure.

Next, at least one pilot hole 22 is created in the planting media 26 at the base of each plant 30 a certain distance, e.g., 1 inch, from the stalk 90 of the respective plant 30 using the hole punch device 14. Importantly, each pilot hole 22 is created such that each respective pilot hole 22 extends into the root zone 34, i.e., into the roots 38, of the respective plant 30, as indicated at 204. Again, it should be understood that the order of configuring the egg solution injection system 18 and then creating the pilot hole(s) 22 is merely exemplary and that the order could be reversed and remain within the scope of the disclosure. That is, in various embodiments, the pilot hole(s) 22 can be created first and then the egg solution injection system 18 is configured. It should also be understood that it is envisioned that one, two or more pilot holes 22 can be created and remain within the scope of the present invention. Creating a plurality of pilot holes 22 around the stalk 90 of each plant 30 will increase the infestation rate for the respective plant 30. For example, in various embodiments, a pair of opposing pilot holes 22 are created, e.g., a first pilot hole 22 and a second pilot hole 22 on the opposing side of each plant stalk 90, wherein each pilot hole 22 extends into the root zone 34 of the respective plant 30.

Once the egg solution injection system 18 has been configured and the pilot hole(s) 22 created, the tip 86 of the injection gun dispensing wand 82 is sequentially placed in close proximity to, or slightly protruding into, the base 22A of each pilot hole 22. The injection gun trigger mechanism 70 is then activated to dispense the selected metered dosage of the multi-tiered hatching egg solution at the selected pressure into the respective pilot hole 22. More specifically, as a result of the selected pressure, the dispensed selected metered dosage is injected into the respective pilot hole 22 such that at least a portion, e.g., a substantial portion, of the multi-tiered hatching egg solution is deposited into the root zone 34 of the respective plant 30, as indicated at 206. The base 22A of each pilot hole 22 is then closed to prevent the egg solution from dehydrating by pushing some of the surrounding planting media 26 over the pilot hole base 22A, as indicated at 208.

Referring now to FIG. 1, the plant root infestation system 10 further includes a chassis frame 122 structured and operable to support and removably retain at least the egg solution container(s) 42 and the propellant container(s) 46, and, in various implementations, the injection gun 58 and/or the hole punch device 14. For example, in various embodiments, the plant root infestation system 10 can include a portable chassis frame 122 structured and operable to be carried by an operator, e.g., mounted on the operator's back like a backpack, and to support and removably retain the egg solution container(s) 42 and the propellant container(s) 46. In such embodiments, the injection gun 58 and the hole punch device 14 can be hand held devices manually operated by the operator to create the pilot hole(s) 22 and activate the injection gun trigger device 70. Further, in such embodiments, upon manual activation of the trigger device 70, the selected dosage is automatically dispensed from the dispensing wand tip 86 at the selected regulated pressure, via the dosage regulator 74 and the pressure regulator 50, as described above.

Alternatively, in various embodiments, the plant root infestation system 10 can include a mobile chassis frame 122 structured and operable to be either self-propelled or pulled by a vehicle and to support and removably retain the egg solution container(s) 42, the propellant container(s) 46, the injection gun 58 and the hole punch device 14. In such embodiments, the injection gun 58 and hole punch device 14 can be automatically operated to create the pilot hole(s) 22 and activate the injection gun trigger device 70. Further, in such embodiments, upon automated activation of the trigger device 70 the selected dosage is automatically dispensed from the dispensing wand tip 86 at the selected regulated pressure, via the dosage regulator 74 and the pressure regulator 50, as described above.

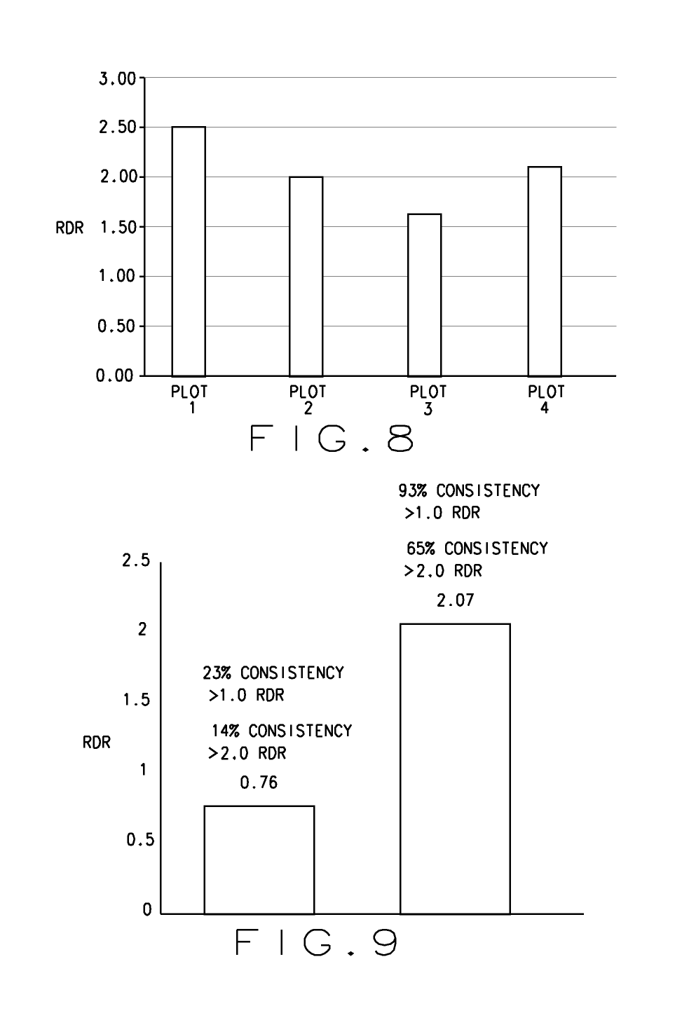

Referring now to FIGS. 8 and 9, the plant root infestation system 10, as described above was utilized to infest four test plots of corn plants 30, wherein each test plot included approximately 4,000 standard corn plants, i.e., non-insecticidal hybrids and/or inbreds that have not been treated with an insecticide. The egg solution comprised western corn rootworm and the concentration of eggs in the egg solution was approximately 250 eggs per milliliter of suspension solution. The injection pressure was set to approximately 30 psi, via the pressure regulator 50, and the metered dosage was set to approximately 5 milliliters. Additionally, via the hole punch device 14, a first pilot hole 22 was created adjacent a first side of each plant stalk 90 and a second pilot 22 hole was created adjacent a second side of each plant stalk 90, opposite the first pilot hole 22. The pilot holes 22 were created at approximately 45° relative to the planting media surface 26A and extended into the respective root zone 34 to substantially form a V pattern. Furthermore, a two-tiered hatching solution was utilized wherein 50% of the eggs were non-incubated and 50% of the eggs were incubated for five days.

FIG. 8 illustrates the Root Damage Rates (RDR), utilizing the Iowa State Rating System, for the four test plots ten weeks after application of the egg solution utilizing the plant root infestation system 10 as described above. For example, the results shown in FIGS. 8 and 9 utilizing the following Rating System: 1) Node-injury scale: 0.01-0.09 has a description of “feeding scars and tracks (discolored, brown areas on the root) can be viewed”, and an impact classified as “non-economic”; 2) Node-injury scale: 0.1-0.9 has a description of “root pruning occurs but less than a full node”, and an impact classified as “damage may be economic under certain conditions”; 3) Node-injury scale: 1.0-1.09 has a description of “at least a full node (or equivalent) destroyed within 4 inches of the plant”, and an impact classified as “probably economic unless conditions for root growth are favorable after damage, lodging is minimal”; 4) Node-injury scale: 2.0-2.9 has a dscription of “two or more nodes are gone”, and an impact classifies as “severe, lodging and goose-necking are common”; and 5) Node-injury scale: 3.0 has a description of “three or more nodes are gone”, and an impact classified as “devastating, logging and goose-necking almost certain”.

As shown in FIG. 8, test plot 1 yielded a RDR of approximately 2.50, test plot 2 yielded a RDR of approximately 2.00, test plot 3 yielded a RDR of approximately 1.65, and test plot 4 yielded a RDR of approximately 2.13. Hence, as shown in FIG. 9, the average RDR of the four test plots due to western corn rootworm infestation evoked utilizing the plant root infestation system 10, as described above, was approximately 2.07. Subsequent field testing yielded an average RDR of approximately 1.62 for additional test plots due to western corn rootworm infestation evoked utilizing the plant root infestation system 10, as described above. FIG. 9 also illustrates an average RDR of approximately 0.76 for four substantially similar test plots infested with western corn rootworm utilizing the known method of pulling a blade through the planting media along a row of plants 30 to create a furrow adjacent the row of plants 30 and then depositing the egg solution into the furrow. Hence, utilizing the plant root infestation system 10, as described above, to infest corn plants provides a substantial increase in the RDR, e.g., approximately 1.31, over at least one known system and method for infestation.

FIG. 9 also illustrates that approximately 65% of the corn plants infested via the plant root infestation system 10 yielded a RDR of greater than 2.00 (i.e., a consistency rate of >2.0 RDR), which is approximately 51% higher than that yielded using the known system and method. Still further, FIG. 9 illustrates that approximately 93% of the corn plants infested via the plant root infestation system 10 yielded a RDR of greater than 1.00 (i.e., a consistency rate of >1.0 RDR), which is approximately 70% higher than that yielded using the known system and method.

The plant root infestation system 10 can be utilized to infest one or more plants 30, as described above, for testing the resistance or susceptibility of any plant to any selected larval insect. For example, the plant root infestation system 10 can be utilized in plant breeding to test the resistance of various insecticidal hybrid and/or inbred plants, e.g., insecticidal hybrid and/or inbred corn plants, to a particular larval insect, e.g., western corn rootworm. Or, for example, the plant root infestation system 10 can be utilized in plant breeding to test the efficacy of various insecticide treatments to a particular larval insect.

The description herein is merely exemplary in nature and, thus, variations that do not depart from the gist of that which is described are intended to be within the scope of the teachings. Such variations are not to be regarded as a departure from the spirit and scope of the teachings.

What is claimed is:

1. A method for infesting the roots of a plant with larval insects, said method comprising injecting an egg solution into a root zone of the plant, the root zone disposed within a planting media of the plant.

2. The method of claim 1, wherein injecting the egg solution into the root zone of the plant comprises formulating the egg solution to comprise a plurality of insect eggs suspended in a solution, wherein the plurality of eggs includes a first portion of eggs that have been incubated for a first period of time and a second portion of eggs that have been incubated for a second period of time.

3. The method of claim 2, wherein one of the first and the second periods of time comprises substantially zero seconds such that the respective portion of the eggs are non-incubated.

4. The method of claim 1, wherein injecting the egg solution into the root zone comprises:
creating at least one pilot hole in the planting media such that the at least one pilot hole extends into the root zone of the plant; and
injecting the egg solution into the at least one pilot hole such that the egg solution is deposited within the root zone.

5. The method of claim 4, wherein creating the at least one pilot hole comprises:
pushing a head of a hole punch device into the planting media such that at least a portion of the hole punch head extends into the root zone; and
pulling the hole punch head out of the planting media such that the at least one pilot hole is created.

6. The method of claim 4, wherein creating the at least one pilot hole comprises creating a conical shaped pilot hole wherein a diameter of a base of the at least one pilot hole at a surface of the planting media is larger than a diameter of a distal end of the at least one pilot hole disposed within the root zone.

7. The method of claim 4, wherein creating the at least one pilot hole that extends into the root zone of the plant comprises creating an angled pilot hole such that a base of the pilot hole at a surface of the planting media is a certain distance away from a stalk of the plant and the hole extends downward and radially inward into the planting media such that at least a distal portion of the pilot hole is disposed within the root zone.

8. The method of claim 7, wherein creating the at least one pilot hole that extends into the root zone comprises creating a plurality of angled pilots about the stalk of the plant wherein at least two of the angled pilot holes are created on opposing sides of the stalk and form a V shape in the planting media, each angled pilot hole extending into the root zone.

9. The method of claim 7, wherein creating the at least one pilot hole that extends into the root zone comprises creating a plurality of angled pilots about the stalk of the plant wherein at least two of the angled pilot holes are created on opposing sides of the stalk and form a X shape in the planting media, each angled pilot hole extending into the root zone.

10. The method of claim 4, wherein injecting the egg solution into the at least one pilot hole such that the egg solution is deposited within the root zone comprises:
placing a tip of a dispensing wand of an injection gun of an automated egg solution injection system in close proximity to a base of the at least one pilot hole at a surface of the planting media; and
dispensing the egg solution from the tip of the dispensing wand into the at least one pilot hole.

11. The method of claim 9, wherein injecting the egg solution into the at least one pilot hole such that the egg solution is deposited within the root zone comprises dispensing the egg solution into the at least one pilot hole at a regulated pressure using the injection gun and a pressure regulator of the automated egg solution injection system such that at least a substantial quantity of the injected egg solution is deposited in the root zone.

12. The method of claim 11, wherein injecting the egg solution into the at least one pilot hole at regulated pressure comprises closing the tip of the injection gun dispensing wand, via a check valve disposed at the tip of the dispensing wand, after the egg solution is injected into the at least one pilot hole.

13. The method of claim 4, wherein injecting the egg solution into the at least one pilot hole such that the egg solution is deposited within the root zone comprises injecting a metered dosage of the egg solution into the at least one pilot hole using an injection gun and a dosage regulator of an automated egg solution injection system such that a predetermined amount of the egg solution is injected into the pilot hole.

14. A method for infesting the roots of a plant with larval insects, said method comprising injecting an egg solution into a root zone of the plant, the root zone disposed within a planting media of the plant, wherein the egg solution includes a plurality of insect eggs suspended in a solution, the egg solution having a first portion of eggs that have been incubated for a first period of time and a second portion of eggs that have been incubated for a second period of time.

15. The method of claim 14, wherein one of the first and the second periods of time comprises substantially zero seconds such that the respective portion of the eggs are non-incubated.

16. The method of claim 14, wherein injecting the egg solution into the root zone comprises:
creating at least one pilot hole in the planting media such that the at least one pilot hole extends into the root zone of the plant; and
injecting the egg solution into the at least one pilot hole such that the egg solution is deposited within the root zone.

17. The method of claim 16, wherein creating the at least one pilot hole comprises:
pushing a head of a hole punch device into the planting media such that at least a portion of the hole punch head extends into the root zone; and
pulling the hole punch head out of the planting media such that the at least one pilot hole is created.

18. The method of claim 16, wherein creating the at least one pilot hole comprises creating a conical shaped pilot hole wherein a diameter of a base of the at least one pilot hole at a surface of the planting media is larger than a diameter of a distal end of the at least one pilot hole disposed within the root zone.

19. The method of claim 16, wherein creating the at least one pilot hole that extends into the root zone of the plant comprises creating an angled pilot hole such that a base of the pilot hole at a surface of the planting media is a certain distance away from a stalk of the plant and the hole extends downward and radially inward into the planting media such that at least a distal portion of the pilot hole is disposed within the root zone.

20. The method of claim 19, wherein creating the at least one pilot hole that extends into the root zone comprises creating a plurality of angled pilots about the stalk of the plant wherein at least two of the angled pilot holes are created on opposing sides of the stalk and form a V shape in the planting media, each angled pilot hole extending into the root zone.

21. The method of claim 19, wherein creating the at least one pilot hole that extends into the root zone comprises creating a plurality of angled pilots about the stalk of the plant wherein at least two of the angled pilot holes are created on opposing sides of the stalk and form a X shape in the planting media, each angled pilot hole extending into the root zone.

22. The method of claim 16, wherein injecting the egg solution into the at least one pilot hole such that the egg solution is deposited within the root zone comprises:
placing a tip of a dispensing wand of an injection gun of an automated egg solution injection system in close proximity to a base of the at least one pilot hole at a surface of the planting media; and
dispensing the egg solution from the tip of the dispensing wand into the at least one pilot hole.

23. The method of claim 22, wherein injecting the egg solution into the at least one pilot hole such that the egg solution is deposited within the root zone comprises dispensing the egg solution into the at least one pilot hole at regulated pressure using the injection gun and a pressure regulator of the automated egg solution injection system such that at least a substantial quantity of the injected egg solution is deposited in the root zone.

24. The method of claim 23, wherein injecting the egg solution into the at least one pilot hole at regulated pressure comprises closing the tip of the injection gun dispensing wand, via a check valve disposed at the tip of the dispensing wand, after the egg solution is injected into the at least one pilot hole.

25. The method of claim 16, wherein injecting the egg solution into the at least one pilot hole such that the egg solution is deposited within the root zone comprises injecting a metered dosage of the egg solution into the at least one pilot hole using an injection gun and a dosage regulator of an automated egg solution injection system such that a predetermined amount of the egg solution is injected into the pilot hole.

26. A method for infesting the roots of a plant with larval insects, said method comprising:
depositing a quantity of an egg solution in an egg solution supply container of an automated egg solution injection system, the egg solution including a plurality of insect eggs suspended in a solution, the egg solution having a first portion of eggs that have been incubated for a first period of time and a second portion of eggs that have been incubated for a second period of time;
creating at least one pilot hole in a planting media from which the plant is growing such that the at least one pilot hole extends into a root zone of the plant, the root zone disposed within the planting media;
placing a tip of a dispensing wand of an injection gun of the automated egg solution injection system in close proximity to a base of the at least one pilot hole at a surface of the planting media, the injection gun and dispensing wand fluidly connected to the egg solution supply container; and
dispensing a quantity of the egg solution from the tip of the dispensing wand into the at least one pilot hole at a regulated pressure using a pressure regulator of the automated egg solution injection system such that the egg solution is injected into the at least one pilot hole and at least a substantial portion of the injected egg solution is deposited in the root zone.

27. The method of claim 26, wherein one of the first and the second periods of time comprises substantially zero seconds such that the respective portion of the eggs are non-incubated.

28. The method of claim 26 wherein creating the at least one pilot hole comprises:
pushing a head of a hole punch device into the planting media such that at least a portion of the hole punch head extends into the root zone; and
pulling the hole punch head out of the planting media such that the at least one pilot hole is created.

29. The method of claim 26, wherein creating the at least one pilot hole comprises creating a conical shaped pilot hole wherein a diameter of a base of the at least one pilot hole at a surface of the planting media is larger than a diameter of a distal end of the at least one pilot hole disposed within the root zone.

30. The method of claim 26, wherein creating the at least one pilot hole that extends into the root zone of the plant comprises creating an angled pilot hole such that a base of the pilot hole at a surface of the planting media is a certain distance away from a stalk of the plant and the hole extends downward and radially inward into the planting media such that at least a distal portion of the pilot hole is disposed within the root zone.

31. The method of claim 30, wherein creating the at least one pilot hole that extends into the root zone comprises creating a plurality of angled pilots about the stalk of the plant wherein at least two of the angled pilot holes are created on opposing sides of the stalk and form a V shape in the planting media, each angled pilot hole extending into the root zone.

32. The method of claim 30, wherein creating the at least one pilot hole that extends into the root zone comprises creating a plurality of angled pilots about the stalk of the plant wherein at least two of the angled pilot holes are created on opposing sides of the stalk and form a X shape in the planting media, each angled pilot hole extending into the root zone.

33. The method of claim 26, wherein dispensing a quantity of the egg solution from the tip of the dispensing wand into the at least one pilot hole at a regulated pressure comprises closing the tip of the injection gun dispensing wand, via a check valve disposed at the tip of the dispensing wand, after the egg solution is injected into the at least one pilot hole.

34. The method of claim 26, wherein dispensing a quantity of the egg solution from the tip of the dispensing wand into the at least one pilot hole at a regulated pressure comprises injecting a metered dosage of the egg solution into the at least one pilot hole using the injection gun and a dosage regulator of the automated egg solution injection system such that a predetermined amount of the egg solution is injected into the pilot hole.

35. A system for infesting the roots of a plant with larval insects, said system comprising:
a hole punch device structured and operable to create at least one pilot hole in a planting media from which the plant is growing such that the at least one pilot hole extends into a root zone of the plant, the root zone disposed within the planting media; and
an automated egg solution injection system structured and operable to inject an egg solution into the at least one pilot hole such that at least a substantial portion of the egg solution is deposited into the root zone of the plant, the automated egg solution injection system comprising:
at least one egg solution supply container structured to retain a quantity of the egg solution;
the egg solution comprising a plurality of insect eggs suspended in a solution, the egg solution having a first portion of eggs that have been incubated for a first period of time and a second portion of eggs that have been incubated for a second period of time;
a propellant container structured to retain a quantity of pressurized gas;
a pressure regulator coupled to the propellant container and fluidly connected to the at least one egg solution supply container via a gas conduit, the pressure regulator structured and operable to release the pressurized gas from the propellant container at a regulated pressure, the released gas being directed into the at least one egg solution supply container via the gas conduit;
an injection gun fluidly connected to the at least one egg solution supply container via a solution hose, the injection gun structured and operable to receive the egg solution forced from the at least one egg solution supply container at the regulated pressure by the gas directed from the propellant container into the at least one egg solution supply container at the regulated pressure, and controllably dispense the egg solution at the regulated pressure through a nozzle of the injection gun upon activation of a trigger mechanism of the injection gun;
a dosage regulator coupled to the injection gun nozzle, the dosage regulator structured and operable to receive the egg solution from the injection gun nozzle and dispense a metered amount of the egg solution through an outlet of the dosage regulator; and
a dispensing wand coupled to the dosage regulator outlet, the dispensing wand structured to convey the egg solution from the dosage regulator outlet to a tip of the dispensing wand whereby the metered amount of egg solution is dispensed from the wand tip at the regulated pressure such that egg solution is injected into the at least one pilot hole and deposited in the root zone when the tip of the dispensing wand is place in closed proximity to a base of the hole at the surface of the planting media.

36. The system of claim 35, wherein one of the first period of time the first portion of eggs are incubated and the second period of time the second portion of eggs are incubated comprises substantially zero seconds such that the respective portion of the eggs are non-incubated.

37. The system of claim 35, wherein the hole punch device comprises:
a shaft; and
a head disposed at an end of the shaft, the head structured to create the at least one pilot hole in a planting media when pushed into the planting media.

38. The system of claim 37, wherein the head is structure to have a conical shape such that the at least one pilot hole is created to have a conical shape wherein a diameter of a base of the at least one pilot hole at a surface of the planting media is larger than a diameter of a distal end of the at least one pilot hole disposed within the root zone.

39. The system of claim 35, wherein the dispensing wand comprises a check valve coupled to the tip of the dispensing wand, the check valve structured and operable to close the dispensing wand tip to prevent the egg solution from leaking from of the dispensing wand tip after the metered amount egg solution is dispensed at the regulated pressure.
